(19) United States
(12) Patent Application Publication
(10) Pub. No.: US 2007/0248534 A1
(43) Pub. Date: Oct. 25, 2007

(54) Auger effect-based cancer therapy method

(75) Inventors: Brenda H. Laster, Meitar (IL); Gad Shani, Omer (IL); Moshe Faraggi, Omer (IL); Yuval Golan, Omer (IL)

Correspondence Address: NIXON & VANDERHYE, PC, 901 NORTH GLEBE ROAD, 11TH FLOOR, ARLINGTON, VA, 22203, US

(73) Assignee: Ben-Gurion University of the Negev, Beer Sheva (IL)

(21) Appl. No.: 11/649,795

(22) Filed: Jan. 5, 2007

Related U.S. Application Data
(62) Division of application No. 10/902,277, filed on Jul. 30, 2004.
Application No. 10/902,277 is a continuation-in-part of application No. PCT/IL03/00070, filed on Jan. 29, 2003.

(30) Foreign Application Priority Data
Jan. 30, 2002 (IL) 147898

(51) Int. Cl. A61K 51/12; A61K 103/30

(52) U.S. Cl. 424/1.11

(57) ABSTRACT

A method for the treatment of a tumor, comprising administering to a subject a therapeutically effective amount of a complex of a heavy element with a polydentate, pyrrole-containing macrocyclic ligand substituted with charged chemical groups, wherein said complex is capable of bringing said heavy element into close proximity to the nuclear DNA of cells in said tumors, and irradiating said tumor with photons above the K or L shell adsorption edge of said heavy element to elicit the emission of densely ionizing Auger electrons at the level of DNA.

### FIELD OF THE INVENTION

The present invention relates to the field of radiation therapy. More specifically, the invention provides a radiotherapy method combining brachytherapy with Auger electron therapy.

### BACKGROUND OF THE INVENTION

The general aim of radiotherapy methods is to cause non-repairable damage to the DNA of malignant cells. However, due to the minute size of the DNA relative to the size of the entire cell, only a very small fraction of the radiation applied to the area of a tumor using conventional radiotherapy methods is likely to make contact with, and cause damage to, the DNA itself.

The art has recognized the potential of the Auger effect as a tool for causing severe, non-repairable, biological damage to the DNA of malignant cells. The Auger effect may be defined as the concomitant emission of electrons from the outer shells of an atom upon the removal of an electron from an inner electronic shell. The reason for this phenomena is that the vacancy created in the low-lying orbital (after the first electron has been expelled therefrom) is immediately filled with an electron of higher energy. The energy this releases may result either in the generation of radiation, or in the emission of a second electron. The latter possibility is known as the Auger effect, and the electrons which are sequentially emitted by said effect are named Auger electrons. An element exhibiting the Auger effect is sometimes referred to as an Auger emitter.

Attempts to establish a clinical cancer treatment method based on the Auger effect have met with two main difficulties. The first difficulty is related to the placement of the element exhibiting the effect in close proximity to the targeted DNA. The second difficulty relates to the radiation source required to activate said element to generate the Auger emission.

The potential of Auger electrons to effectively damage the DNA of the malignant cell depends on the localization of the metal atom, from which these electrons are emitted, as close as possible to the DNA. The reason for this is that since the Auger electrons have relatively low energies and high linear energy transfer, their traveling distance in the tissues of the cell is limited to a very short range, between a few nanometers to a few microns. Thus, when these electrons are released from the metal atom, they can damage only those molecules that are situated in the immediate vicinity of said metal.

Feinendegen [Rad. and Environm. Biophys. 12, pp. 85-99 (1975)] discusses various biological applications of the Auger effect. Regarding the infliction of damage to the cellular DNA, the author reports that both iodine and bromine were used as Auger emitters, and that these elements were incorporated into DNA using thymidine analogues (iododeoxyuridine and bromodeoxyuridine, respectively).

Fairchild et al. [Investigative Radiology 17 (4), pp. 407-416 (1982)] describe the generation of Auger electrons from halogen atoms, which were incorporated as analogues of thymidine in DNA. The authors indicate that halogenated deoxyribonucleosides appear to provide Auger electrons having the best available specific access to DNA.

Laster et al. [Radiation Research 133, pp. 219-224 (1993)], also report the use of 5′-iodo-2′-deoxyuridine as a molecular carrier of iodine into the DNA.

Thus, in view of the above, it appears that the art has failed to provide a flexible, broadly applicable method for positioning metals capable of emitting Auger electrons close to the DNA in a cell.

Another critical requirement for therapy methods based on the Auger effect is associated with the radiation source required to activate the Auger emitter. The radiation source must produce a photon capable of ejecting an electron from an inner shell of the metal, thereby triggering the Auger cascade. It may be readily appreciated that in order to increase the number of electrons emitted by the Auger effect, it is most preferable to remove the first electron from the innermost electronic shell of the metal. For example, when the first electron is expelled from the innermost shell (the K shell) of indium, gadolinium and platinum, the number of Auger electrons emitted by said metals are approximately 6, 10 and 16, respectively.

It has been suggested in the art that a radiation source containing a radioactive isotope implanted within a particular body region be used for inducing the emission of Auger electrons from iodine. Such implanted radiation sources are commonly used in a radiotherapy technique known as brachytherapy. However, the requirements for a radioactive isotope to function both as a useful brachytherapy radiation source, and as an efficient activator for the Auger emitter, are not easily met. Specifically, the radioisotope must have an appropriate decay profile and, in addition, it must be easily encapsulated within available casings, to form the “brachytherapy seed” (this term is used in the art to define the small canister, containing the radioactive isotope). The commercially available brachytherapy seeds contain radioactive isotopes (iodine-125, palladium-103 and iridium-192) which do not have the energy output required to activate the above-mentioned potential Auger emitters (indium, gadolinium and platinum). An additional drawback associated with said commercially available brachytherapy seeds is their relatively short half-lives. The valuable properties of samarium-145 (which was disclosed in US Statutory Invention Registration no. H669 as a radiation source useful both for brachytherapy applications and for the activation of iodine as Auger emitter) have not been exploited, since the art has failed to provide a successful method for densely packaging the same in suitable canisters, in order to permit its utilization as a radiation source in radiotherapy.

It is therefore an object of the present invention to provide an Auger effect-based cancer therapy method, allowing the Auger emitter to be placed in close proximity to the target DNA, and the subsequent activation of the Auger emitter by means of effective radiation sources.

### SUMMARY OF THE INVENTION

The inventors have surprisingly found that pyrrole-containing compounds, and specifically, porphyrins, which are substituted with charged organic groups, may be used to position heavy elements in very close proximity to the DNA in tumor cells, such that, following the irradiation of said tumor cells using a suitable radiation source, said DNA is severely damaged.

The inventors have also found that it is possible to significantly increase the damage caused to the DNA in tumor cells by applying radiation at the tumor zone, said radiation including photons that are capable of inducing the heavy element to emit Auger electrons, that is, photons preferably having energy above the K-shell energy of said heavy element. The inventors believe that irradiating the tumor zone with such photons induces the heavy element to emit Auger electrons, which, due to the unexpectedly small distance between said heavy element and the DNA, contribute to the severe destruction of said DNA.

The inventors have also found that the energy required to activate particularly important potential Auger emitters such as In, Gd, Pt, Au and Pd may be provided by a radiation source containing suitable radioactive isotopes that are implanted at the site to be treated. Thus, this aspect of the present invention combines the utility of radiation sources for brachytherapy with the activation of Auger emitters that are located in close proximity to the DNA in the tumor cell.

According to one aspect, the invention provides a method for the treatment of a tumor, comprising administering to a subject a therapeutically effective amount of a complex of a heavy element with a polydentate, pyrrole-containing macrocyclic ligand substituted with charged chemical groups, wherein said complex is capable of bringing said heavy element into close proximity to the nuclear DNA of cells in said tumor, and irradiating said tumor.

As used herein, the term “heavy element” refers to any chemical element, which, following suitable activation, is capable of exhibiting the Auger effect. These elements generally have an atomic number between 35 and 85. Preferably, the heavy element used according to the present invention is selected from the group consisting of: In, Gd, Pt, Ru, Os, Au, La, Ce, Ba, Cs, I, Te, Sb, Sn, Cd, Ag and Pd. Most preferred are metals such as indium, gadolinium, platinum, palladium and gold.

The term “polydentate, pyrrole-containing macrocyclic ligand” as used herein, refers to a molecule with pyrrole rings that are fused together to form a macrocyclic structure. In a preferred embodiment of the present invention, the polydentate, pyrrole-containing macrocyclic ligand is selected from the group consisting of porphyrin or phthalocyanine compounds. Thus, the therapeutic agent according to the present invention is provided in the form of a complex in which a heavy element, which is a metal capable of exhibiting the Auger effect, as defined above, is coordinated with a polydentate, pyrrole-containing macrocyclic ligand substituted with charged chemical groups.

The term “close proximity”, as used herein, indicates that the distance between the heavy element-containing complex and the nuclear DNA of cells in the tumor is less than the traveling distance of Auger electrons that may be generated by said heavy element. Preferably, this distance is less than 100 nm, and more preferably, less than 50 nm. Particularly preferred complexes according to the present invention are those that can bind to the nuclear DNA of cells in the tumor.

The polydentate, pyrrole-containing macrocyclic ligand is substituted with charged organic groups, which are preferably positively charged quaternary ammonium groups or negatively charged carboxylic acid residues.

Preferred positively charged quaternary ammonium groups are represented by the following formula: 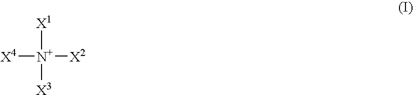
wherein X¹, X², X³ and X⁴ are independently selected from the group consisting of substituted or unsubstituted C₁-C₅ alkyl, C₂-C₅ alkenyl, C₂-C₅ alkynyl, C₃-C₈ carbocyclic radicals, aryl radicals, heterocyclic radicals, heteroaryl radicals, or X¹ and X² are taken together with the nitrogen atom to which they are connected to form a heterocyclic radical or heteroaryl radical, wherein, in case of the latter radical, X⁴ is absent. The above substituent of formula (I) is linked to the polydentate, pyrrole-containing macrocyclic ligand (e.g., the porphirin system) via any of the substituents X¹, X², X³ and X⁴, as illustrated herein below.

According to one variant of the invention, X¹ and X² are taken together with the nitrogen atom to which they are connected to form a heteroaryl radical, and most preferably a heteroaryl selected from the group consisting of pyridine and quinoline, X³ is C₁-C₅ alkyl and X⁴ is absent. Particularly preferred are quaternary ammonium groups that are N-alkyl-4-pyridyls represented by the following formula: 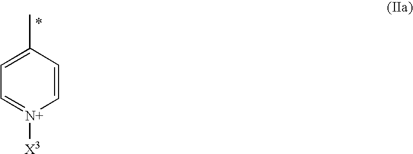
wherein X³ is a straight or branched C₁-C₅ alkyl. The chemical bond indicated by asterisk signifies the linkage to the porphyrin system.

According to another embodiment of the invention, preferred quaternary ammonium groups of formula I are those wherein X⁴ is aryl group, most preferably phenyl, as represented by the formula below: 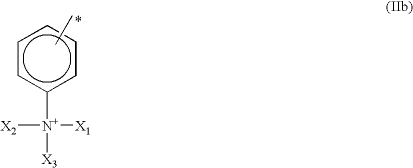
wherein X¹, X² and X³ are straight or branched C₁-C₅ alkyl, and wherein the chemical bond indicated by asterisk signifies the linkage to the porphyrin system, which is preferably via the para position.

According to a preferred embodiment of the invention, the polydentate, pyrrole-containing macrocyclic ligand is substituted with hydrophobic moieties that are selected from the group consisting of straight or branched C₁-C₅ alkyl chains, C₃-C₈ cycloalkyl or aliphatic structures such as fullerene (C₆₀₋). According to a particularly preferred embodiment of the present invention, the hydrophobic moieties are provided by the X¹, X², X³ and X⁴ attached to the quaternary ammonium of formula I. According to another preferred embodiment of the invention, the hydrophobic moieties are linked to the polydentate, pyrrole-containing macrocyclic ligand through a linker provided by a carboxylic acid residue.

In a preferred embodiment of the present invention, the heavy element-containing complex is metallo-porphyrin represented by the structure of formula III: 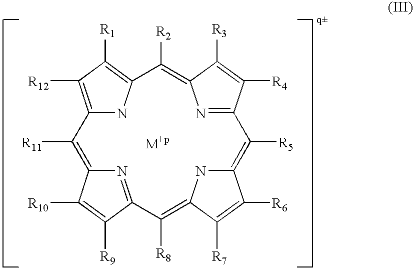
wherein M^{p+} designates a cation of the heavy element capable of exhibiting the Auger effect, which is preferably selected from the group consisting of indium, gadolinium, platinum and gold, q± represents the total charge of the complex, which may be either positive or negative, and wherein:

(i) R₂, R₅, R₈ and R₁₁ are positively charged N-alkyl pyridyl groups of formula IIa above, and R₁, R₃, R₄, R₆, R₇, R₉, R₁₀ and R₁₂ are hydrogen (that is, the heavy element-containing complex of formula III belongs to the class of metallo-tetra(N-alkyl-4-pyridyl)porphyrins); or

(ii) R₂, R₅, R₈ and R₁₁are positively charged N,N, N-trialkyl anillinium of formula IIb above, and R₁, R₃, R₄, R₆, R₇, R₉, R₁₀ and R₁₂ are hydrogen; or

(iii) R₃, R₆, R₁₀ and R₁₂ are methyl groups, R₇ and R₉ are negatively charged carboxylic acid residues —(CH₂)n C(O)O⁻, wherein n is an integer between 1-5, R₁ and R⁴ are represented by the formula 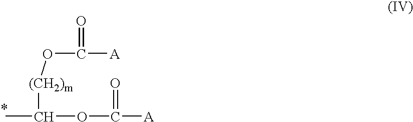
wherein m is an integer between 1-5 and A is a hydrophobic moiety as defined above, and preferably, fullerene (C₆₀), and R₂, R₅, R₈ and R₁₁ are hydrogen. The chemical bond indicated by asterisk signifies the linkage to the porphyrin system.

Most preferably, the heavy element-containing complex is selected from the group consisting of:

A complex of formula III (i) above [that is, the class of metallo-tetra(N-alkyl-4-pyridyl)porphyrins], wherein M is preferably In³⁺ or Pt²⁺, and each of R₂, R₅, R₈ and R₁₁is N-methyl 4-pyridyl, and R₁, R₃, R₄, R₆, R₇, R₉, R₁₀ and R₁₂ are hydrogen ([In³⁺-tetra(N-methyl-4-pyridyl)-porphyrin]⁵⁺ and [Pt²⁺-tetra(N-methyl-4-pyridyl)-porphyrin]⁴⁺, respectively).

A complex of formula III(ii) above, wherein M is In³⁺, each of R₂, R₅, R₈ and R₁₁, is N,N,N-trimethyl anillinium, and R₁, R₃, R₄, R₆, R₇, R₉, R₁₀ and R₁₂ are hydrogen [In³⁺-tetra(4-N,N,N-trimethylanillinium)porphyrin]⁵⁺.

A complex of formula III(iii) above, wherein M is In³⁺, R₇ and R₉ are (CH₂)₂—C(O)O⁻, m is 2 and A is fullerene [In³⁺-tetrakis-fullerene-carboxylate esters of 2,4 bis α,β-dihydroxyethyl)-deutroporphyrin IX]¹⁻.

According to a particularly preferred embodiment of the invention, the tumor region is irradiated by means of a radiation source having an energy output capable of activating the heavy element to emit Auger electrons therefrom. Most preferably, the radiation source produces a photon (x- or γ-ray), the energy of which is above the M-, L- or K-shell energies of said heavy element.

According to a particularly preferred embodiment of the invention, the radiation source is implanted near or in the body region to be treated, said radiation source comprising one or more radioactive isotopes generating the desired energy for removing the primary electron from an inner electronic shell of said heavy element, wherein said one or more radioisotopes are encapsulated within a casing, which is preferably in the form of a closed, cylindrically shaped canister. Thus, in a preferred embodiment of the invention, the radiation source simultaneously functions as a brachytherapy source (seed) and as an activator for the Auger emitter.

In a preferred embodiment of the invention, the radiation source comprises one or more radioactive isotopes generating photons having energies in the range of 25 to 100 keV, said isotopes having half-lives longer than 20 days. Preferably, said isotopes are selected from the group consisting of ¹⁰³Pd, ¹⁴⁵Sm, ¹⁷⁰Tm, ¹²⁵I, a mixture of ¹²⁵I and ¹²⁷I, ²³⁴Th, ^{93m}Nb, ¹⁴⁰Ba, ¹⁹⁵Au, ¹⁴⁴Ce, ^{125m}Te, ^{95m}Tc, ²⁴⁵Am, ²⁵³Eu, ¹⁸³ Re, ¹⁸⁵W, ¹⁵⁹Dy, ^{127m}Te, ¹⁶⁹Yb, ¹⁰⁵Ag, ^{119m}Sn, ¹⁷¹Tm, ¹⁴⁵Pm, ¹⁵³Gd, ¹³³Ba, ¹⁷⁴Ln, ¹⁶³Tm, ¹⁴⁷Eu, ¹⁷⁵Hf and ^{97m}Tc. The radioactive isotope is packed within a canister (“brachytherapy seed”), which is preferably made of titanium.

In another aspect, the invention provides a radiation source (e.g., a brachytherapy seed) comprising a mixture of ¹²⁵I and ¹²⁷I. The inventors have found that a mixture of the radioactive isotope of iodine, ¹²⁵I, with non-radioactive iodine, ¹²⁷I, possesses valuable energy emission features useful in relation to the activation of indium to emit Auger electrons. Specifically, it has been found that a mixture of ¹²⁵I and ¹²⁷I emits x-ray radiation at energy of 28.6 keV, in addition to the emission spectrum of the radioactive isotope ¹²⁵I, which consists mostly of x-ray emitted at energy of 27.5 keV. The x-ray photon having energy of 28.6 keV is capable of removing an electron from the K-shell of indium, the binding energy of which being 27.9 keV.

In another particularly preferred embodiment of the present invention, the implanted radiation source comprises ¹⁷⁰Tm. The inventors have found that ¹⁷⁰Tm exhibits several useful properties, emitting a γ-ray of energy 84.4 keV and an x-ray of energy 52.4 keV and having a relatively long half life of 130 days. These properties, in addition to the fact that ¹⁷⁰Tm may be easily and effectively loaded within a suitable canister, permit the combined use of said isotope as a brachytherapy source and as an activator for the particularly useful Auger emitters platinum and gadolinium, which have K-shell energies of 78.4 keV and 50.24 keV, respectively.

In another preferred embodiment of the present invention, the radiation source is provided by a canister, which is preferably a titanium canister, enclosing radioactive ¹⁴⁵Sm, wherein said samarium-containing canister is prepared by a method comprising the steps of providing a solution containing samarium ions, positioning a working electrode and at least one counter electrode in contact with said solution, connecting said working electrode and said at least one counter electrode to the negative and positive poles of a power source, respectively, passing an electrical current between said electrodes to electrochemically deposit elemental samarium on said working electrode in a geometrical form corresponding to the form of the interior of the canister, and concurrently or sequentially loading said canister with said elemental samarium. Subsequently, the ¹⁴⁴Sm is neutron-irradiated to produce the radioactive ¹⁴⁵Sm.

In another aspect, the present invention provides a therapeutic composition comprising a complex of a heavy element with a polydentate, pyrrole-containing macrocyclic ligand substituted with charged chemical groups, together with a pharmaceutically acceptable carrier, for use in radiation therapy of tumors.

In another aspect, the present invention relates to the use of a complex of a heavy element with a polydentate, pyrrole-containing macrocyclic ligand substituted with charged chemical groups, for the preparation of a medicament useful in radiation therapy of tumors.

In another aspect, the present invention provides a therapeutic system suitable for the radiation therapy of tumors, said therapeutic system comprising:

a therapeutic composition comprising a complex of a heavy element with a polydentate, pyrrole-containing macrocyclic ligand substituted with charged chemical groups; and

a radiation source to irradiate said tumor.

According to a preferred embodiment of the invention, the radiation source has an energy output capable of activating the heavy element to emit Auger electrons therefrom. Preferably, the radiation source is provided in the form of a radioactive isotope packed in implantable, cylindrically shaped, canister.

Another preferred embodiment of the present invention relates to Auger radiation therapy method based on the use of Pt²⁺-tetra (N-methyl-4-pyridyl)-porphine as the heavy element-containing complex in combination with palladium-103 brachytherapy seed. It has been surprisingly found that Pt can be effectively activated at its L absorption edges to elicit Auger emission by means of said palladium-103 brachytherapy seed. The invention also provides a therapeutic composition comprising Pt²⁺-tetra (N-methyl-4-pyridyl)-porphine for use in Auger radiation therapy.

### BRIEF DESCRIPTION OF THE DRAWINGS

FIG. 1 illustrates the preferential localization of the In³⁺-tetra (N-methyl-4-pyridyl)-porphyrin) complex within the nucleus.

FIG. 2 illustrates the binding of the In³⁺-tetra(N-methyl-4-pyridyl)-porphyrin) complex to the DNA.

FIG. 3 shows the energy spectrum of thulium-170 seed.

FIG. 4 illustrates the combination of Pt²⁺-tetra(N-methyl-4-pyridyl)-porphyrin) complex within ¹⁰³Pd brachytherapy seeds.

### DETAILED DESCRIPTION OF PREFERRED EMBODIMENTS

The method for treating cancer according to the present invention involves the administration to a subject of a therapeutic agent, which is a complex containing a heavy element attached to a polydentate, pyrrole-containing macrocyclic ligand, in order to position the heavy element in close proximity to the DNA of the cell of the tumor, and the irradiation of said tumor, to cause non-repairable damage to the DNA.

Definitions

As used throughout this specification and claims, the following terms have the meanings specified.

The term “tumor” as used herein, refers to both malignant and benign tumors. Tumors that may be treated according to the present invention are particularly tumors that are accessible for the implantation of brachytherapy seeds. Examples of such tumors are prostate cancer, breast cancer, brain cancer, melanoma, head and neck and sarcoma.

The term “alkyl” refers to a monovalent group derived from a straight or branched chain saturated hydrocarbon of 1 to 5 carbon atoms, by the removal of a single hydrogen atom and include, for example, methyl, ethyl, n- and iso-propyl, and the like.

The term “alkenyl”, as used herein, refers to monovalent straight or branched chain groups of 2 to 5 carbon atoms containing one or more carbon-carbon double bonds, derived from alkene by the removal of one hydrogen atom and include, for example, ethenyl, 1-propenyl, 2-propenyl, and the like.

The term “alkynyl”, as used herein, refers to monovalent straight or branched chain groups of 2 to 5 carbon atoms containing one or more carbon-carbon triple bond, derived from alkyne by the removal of one hydrogen atom.

The term “carbocyclic radical”, as used herein, refers to a monovalent, saturated or partially saturated cyclic hydrocarbon groups such us cyclopropyl, cyclobutyl, cyclopentyl, cyclohexyl, cycloheptyl, and the like.

The term “aryl”, as used herein, refers to substituted or unsubstituted carbocyclic aromatic systems containing one or more fused or non-fused phenyl rings and include, for example, phenyl, naphthyl and the like.

The term “heterocyclic”, as used herein, refers to saturated, partially saturated and unsaturated heteroatom-containing ring-shaped radicals.

The term “heteroaryl”, as used herein, refers to unsaturated heterocyclic radicals, and include, for example, pyridyl. The term also embraces radicals where the heteroaryl radical is fused with aryl radicals, such as, for example, a quinolyl group.

Preparation of the Heavy Element-Containing Complexes

The heavy element-containing complexes to be used according to the invention are either known, or may be prepared, starting from known compounds, by means of methods known in the art. Certain classes of preferred compounds of formula III above are commercially available (Mid-Century Posen, Ill. 60469, USA).

In general, the heavy element-containing complex can be prepared according to the procedures described by Hambright et al. [Inorganic Chemistry, 9(7), pp. 1757-1761 (1970) and Journal of Coordination Chemistry, 12, pp. 297-302 (1983)], wherein an excess of a salt of the heavy element, for example, a chloride salt thereof, is refluxed overnight with the polydentate, pyrrole-containing macrocyclic ligand in an aqueous solution, preferably under acidic conditions. The complex may be precipitated with NaClO₄ or KClO₄. Pharmaceutically acceptable salts of the complex, (e.g., chloride forms), may be prepared by ion-exchange methods. A particularly preferred heavy element-containing complex according to the invention, Platinum (II)-tetrakis(N-methyl-4-pyridyl)porphyrin (in the form of its chloride salt) may be conveniently prepared according to the description of Pasternack et al. [Inorg. Chem. 29, 4483-4486 (1990)].

Representative synthetic procedures for preparing particularly preferred porphyrins suitable for use as polydentate, pyrrole-containing macrocyclic ligands according to the present invention are outlined in the following schemes.

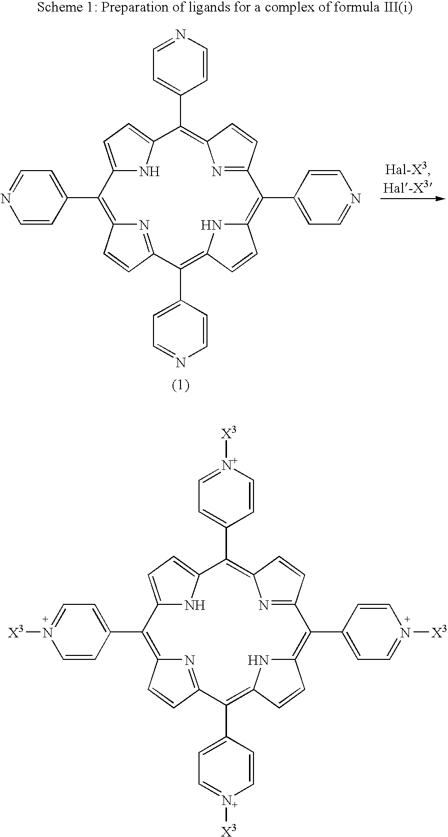

(1) (tetra (4-pyridyl) porphine) is reacted with stoichiometrc amounts of haloalkane Hal-X³ (wherein Hal is Cl, Br or I, most preferably I, and X³ is a straight or branched C₁-C₅ alkyl) in an inert solvent which is preferably DMF or DMSO, under reflux. Optionally, haloalkane Hal′-X³′ (wherein Hal′ is Cl, Br or I, most preferably I, and X³′ is as defined above for X³) is added to the reaction mixture, in order to attach other alkyl groups (X³′≠X³) to the nitrogen atoms of the pyridyl groups. Upon completion of the reaction, the tetra (N-alkyl-4-pyridyl)porphine is separated from the reaction mixture.

The preparation of a particularly preferred ligand according to the present invention, tetra (N-methyl-4-pyridyl)porphine may be accomplished according to the procedure described in Inorganic Chemistry, 9(7), pp. 1757-1761.

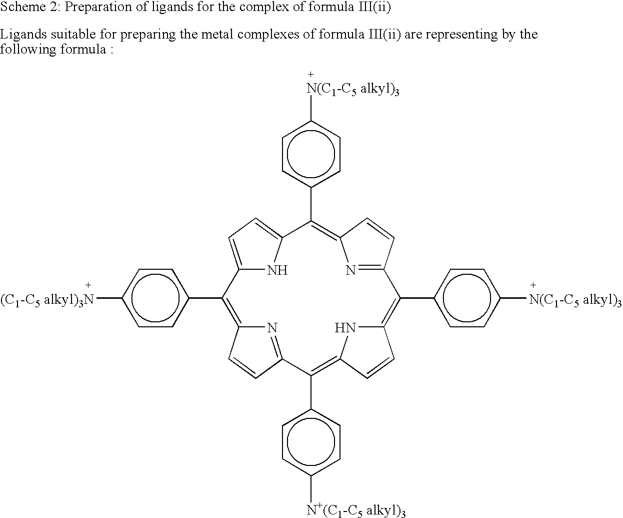

The synthesis of the porphines depicted above may be accomplished according to the procedure described in Indian J. Chem, 15B, pp. 964-966 (1977).

Scheme 3: Preparation of Ligands for the Complex of Formula III(iii)

Ligands suitable for preparing the metal complexes of formula III(iii) may be accomplished according to the procedure described in J. Chem. Soc. Chem. Commun., p. 1769 (1990) for the synthesis of tetrakis-carborane-carboxylate esters of 2,4-bis (α,β-dihydroxyethyl)-deutroporphyrin IX, replacing the carboranes with fullerenes.

Pharmaceutical Compositions

The heavy-element containing complex according to the invention is electrically charged. The complex is administered as a pharmaceutically acceptable salt having suitable counter ions.

The heavy element-containing complexes may be introduced into the subject by any convenient and efficient means. Pharmaceutical compositions that comprise pharmaceutically acceptable salts of said complexes may be specially formulated for local administration or for oral administration.

The term “local administration” includes all possible means for administering the heavy element-containing complexes of the invention at, or close to, the targeted tumor. This term is not limited to syringe injection alone, but also encompasses the use of all commonly used mechanical and electro-mechanical pumping devices, controlled-release devices, infusion systems, and other related mechanisms for local delivery of therapeutic agents.

Injectable preparations suitable for local administration are provided in the form of pharmaceutically acceptable sterile aqueous or non-aqueous solutions, dispersions, suspensions or emulsions as well as sterile powders for reconstitution into sterile injectable solutions or dispersions prior to use. Examples of suitable aqueous or non-aqueous carriers or vehicles include water, Ringer's solution and isotonic sodium chloride solution. Sterile oils may also be employed as a suitable suspending medium.

The injectable formulations can be sterilized, for example, by filtration through a bacterial-retaining filter, or by incorporating sterilizing agents therein.

These formulations may also contain preservatives, wetting agents, emulsifying agents, dispersing agents and surfactants. It is also possible to include osmotically-active agents such as sugars, etc.

Liquid dosage forms for oral administration include pharmaceutically acceptable solutions, emulsions, suspensions and syrups. In addition to the active compounds, the liquid dosage form may contain inert diluents commonly used in the art such as water or other solvents, solubilizing agents and emulsifiers such as ethyl alcohol, isopropyl alcohol, ethyl carbonate, ethyl acetate, propylene glycol and oils. Besides inert diluents, the oral compositions may also include adjuvants such as wetting agents, emulsifying and suspending agents, sweetening, flavoring and perfuming agents.

Solid dosage forms for oral administration include capsules, tablets, pills, powders and granules. In such solid dosage forms, the active compound is mixed with at least one inert, pharmaceutically acceptable excipient or carrier such as sodium citrate or dicalcium phosphate and/or fillers or extenders such as starches, lactose, sucrose, glucose and mannitol, binders such as carboxymethylcellulose and gelatin, humectants such as glycerol, disintegrating agents such as agar-agar, calcium carbonate and potato starch, absorbents and lubricants. The solid dosage forms can be prepared with coatings and shells according to methods known in the art.

Other suitable formulations may be prepared by encapsulating the active ingredient in lipid vesicles or in biodegradable polymeric matrices, or by attaching said active ingredient to monoclonal antibodies. Methods to form liposomes are known in the art. See, for example Liposome Technology (Edited by Gregoriadis G.), CRC Press (1993).

Dosage levels of active ingredients in the pharmaceutical compositions of this invention may be varied so as to obtain an amount of the active complex that is effective to achieve the desired therapeutic response for a particular patient. The selected dosage form will depend on the activity of the particular complex, the route of administration, the severity of the condition being treated and other factors associated with the patient being treated. Typical dose regimes are in the range of 1 to 150 mg/kg.

Radiation Sources

The method for treating tumor according to the present invention involves the irradiation of the tumor site. Preferably, the radiation source used is capable of activating the heavy element that is positioned in close proximity to the DNA in the cells of said tumor, to emit Auger electrons therefrom, in order to increase the damage caused to the DNA. Most preferably, the radiation source produces photons, the energy of said photons being above the binding energy of the electron in the K-shell of the heavy element. The K-shell energy values of various elements are listed in “Table of Isotopes”, by C. M. Lederer and V. S. Shirley, published by Wiley and Sons (1978).

Possible external radiation sources that may be used according to the present invention include synchrotron radiation sources and monochromatic X-ray machines. UV or laser radiation sources may be used as well, as these sources may have a beneficial effect associated with the excitation of porphyrins.

More preferably, an implanted radiation source will be used to activate the heavy element to emit Auger electrons, said radiation source comprising a radioactive isotope packed within a casing, which is preferably in the form of a closed, cylindrically shaped, canister. The typical dimensions of these canisters (“seeds”) are about 0.45 mm in diameter and 0.5 to 1.0 cm in length. The radiation source is prepared by loading the canister, which is preferably made of a material selected from the group consisting of titanium, stainless steel, vanadium, inert bioceramics, glass and porcelain, with the selected radioisotope, and subsequently sealing said canister, preferably by laser welding or other methods known in the art. Suitable techniques include, for example, laser welding, electron beam welding, crimp welding, gas tungsten arc welding, gas metal arc welding, flux cored arc welding, shielded metal arc welding or submerged arc welding.

Modified implantable radiation sources for use in brachytherapy (brachytherapy seeds) and methods for constructing the same are disclosed, for example, in U.S. Pat. No. 6,132,359 and in Chen et al., Med. Phys 28, p. 86-96 (2001), which are incorporated herein entirely by reference. These modified radiation sources may also be used as part of the method of the present invention.

Methods for implanting the radiation sources within the desired body region are well known in the art. In essence, the seeds are implanted according to the geometry of the patient's cancer, in order to ensure that adequate radiation levels reach the tissue. For example, in the case of prostatic cancer, one possible technique involves loading the seeds into the cannula of a needle-like insertion device. Improved techniques for implanting brachytherapy seeds, which may be practiced according to the present invention are disclosed, for example, in U.S. Pat. No. 6,036,632, U.S. Pat. No. 6,267,718 and U.S. Pat. No. 6,311,084. A typical radiation dose can be in the range of 60-70 Gy.

In a preferred embodiment of the present invention, the heavy element contained in the complex is indium, and the implanted radiation source used to irradiate the tumor site comprises a mixture of ¹²⁵I and ¹²⁷I. The radiation source may be prepared by loading small tubes, which are preferably made of titanium, with a mixture of ¹²⁵I and ¹²⁷I. The number of ¹²⁵I atoms required to provide a radiation source having an activity of 1 mCi is 2.8×10¹⁴. Typically, the available volume within the titanium tube is about 1.4×10⁻³ cm³. The total weight of iodine, which may be inserted into said tube is typically about 6.9×10⁻³ g, which corresponds to 3.26×10¹⁹ iodine atoms. The concentration of ¹²⁵I in the mixture is therefore approximately 10⁻⁶ (2.8×10¹⁴/3.26×10¹⁹). In another preferred embodiment of the present invention, the heavy element contained in the complex that is administered to the patient is gadolinium or platinum, and the implanted radiation source used to irradiate the tumor site comprises ¹⁷⁰Tm. The number of ¹⁷⁰ Tm atoms required to provide a radiation source having an activity of 1 mCi is 6×10¹⁴. The radiation source may be prepared by loading small tubes, which are preferably made of titanium, with ¹⁶⁹Tm. Typically, the available volume within the titanium tube is about 1.4×10⁻³ cm³. ¹⁶⁹Tm, preferably in the form of small pieces, is inserted into said tube, whereby a density of about 9.32 g Tm/cm³ can be obtained. The preferred activation time for ¹⁶⁹Tm is about 9.4 days, using a neutron flux of 10¹³ n/cm²·s, or 2.25 hours, using neutron flux of 10¹⁵ n/cm²·s.

In another preferred embodiment of the present invention, the radiation source used to irradiate the tumor site is a ¹⁴⁵Sm-containing canister, which is prepared according to the method described in Israeli patent application no 147199 (PCT/IL02/01013), which is incorporated herein entirely by reference. Briefly, said method comprises the steps of providing a solution containing samarium ions, positioning a working electrode and at least one counter electrode in contact with said solution, connecting said working electrode and said at least one counter electrode to the negative and positive poles of a power source, respectively, passing an electrical current between said electrodes to electrochemically deposit elemental samarium on said working electrode in a geometrical form corresponding to the form of the interior of the canister, and concurrently or sequentially loading said canister with said elemental samarium, and subsequently neutron-irradiating the ¹⁴⁴Sm to produce the radioactive ¹⁴⁵Sm.

Preferably, the solution used for electrodepositing elemental samarium is an aqueous solution of enriched samarium oxide, Sm₂O₃. The preferred concentration of Sm₂O₃ in the aqueous solution is in the range of 10-50 g/liter, and more preferably in the range of 15-25 g/liter. The electrochemical reduction of Sm⁺³ to give elemental samarium is preferably performed under acidic conditions, preferably at a pH in the range of 1.5 to 5, more preferably at a pH in the range of 2 to 3. The pH is preferably adjusted to the desired range by means of nitric acid.

The electrochemical reduction of Sm⁺³ to give elemental samarium is preferably carried out in the presence of a complex-forming anion, which is a ligand capable of forming a complex with Sm⁺³, such that the deposition potential of samarium is reduced, under acidic conditions, and is preferably shifted to a value in the range of −0.50 to −0.80 V vs. SCE (Standard Calomel electrode), and more preferably to a value in the range of −0.60 to −0.70 V vs. SCE. Preferably, the complex-forming anion is selected from the group consisting of the ligands tartrate, oxalate, citrate, EDTA and thiocyanate, most preferably the tartrate ligand. The molar ratio between the complex-forming anion present in the solution and the samarium ion is preferably in the range of 1:1 to 5:1.

Preferably, the electrochemical reduction of Sm⁺³ to give elemental samarium is carried out at a temperature in the range of 25 to 60° C., and more preferably in the range of 30 to 40° C.

Preferably, the electrochemical reduction of Sm⁺³ to give elemental samarium is carried out in a solution containing preservatives and other additives such as brighteners and levelers, which are commonly used in electroplating baths.

According to one of the embodiments described in Israeli patent application no 147199, which is incorporated herein entirely by reference, the counter electrode positioned in the solution may be in the form of a cylindrical grid surface, which is preferably made of a material selected from the group consisting of Pt, platinized Pt or graphite. Preferably, the length and the diameter of said cylindrical surface which constitutes the counter electrode are in the ranges of 7 to 13 cm and 2 to 4 cm, respectively. The working electrode is provided in the form of a wire, which is coaxially positioned within the cylindrical space defined by the counter electrode. Preferably, said wire is made of graphite, although wires made of metals such as Ti may also be used. The diameter of the wire is preferably in the range of 10 to 50 μm.

The working electrode and the counter electrode positioned in the samarium containing solution are electrically connected to the negative and positive poles of a suitable power source, respectively. Typical current density applied according to the process described in Israeli patent application 147199 is in the range of 0.5 to 30 mA/cm², avoiding hydrogen evolution at the cathode. The cylindrical symmetry of the arrangement of the electrodes according to this embodiment of the invention causes the samarium, which is reduced according to the following cathode reaction:
Sm⁺³+3e→Sm
to coat the wire that functions as the working electrode (cathode), such that a solid body made of samarium is obtained, said body having an essentially cylindrical form, wherein the symmetry axis of said body essentially coincides with said wire. Preferably, the diameter of the cross-section of said cylindrical body is about 0.38 mm, such that transverse sections of said cylindrical body can be easily and effectively inserted into a canister intended for use as a brachytherapy seed, said canister typically having an inner cross-section of 0.4 mm. Preferably, the canister is provided in the form of a titanium tube, which is commercially available (Uniform Tubes Inc., South Plainfield, N.J. 07080, USA). Following the packing of elemental samarium inside said tube, the tube is sealed, using the techniques as described hereinabove. The activation of the radiation source may be performed in accordance with the description of US Statutory Invention Registration H669, which is incorporated herein by reference. In general, the strength of the source will vary in accordance with its clinical utility. For example, for brain tumors, a 7 to 10 mCi source will be required to accommodate the larger tumor at the time of diagnosis. Activation of 10¹⁹ atoms of ¹⁴⁴Sm to produce ¹⁴⁵Sm will be accomplished by means of irradiation at a neutron flux of 10¹⁵ neutrons/cm·s, for 15.5 days.

According to an alternative method described in Israeli patent application no 147199, the working electrode is provided in the form of a perforated plate, wherein each hole of said plate contains a titanium tube, the length and the cross-section of said tube being essentially the same as the thickness of said plate and cross-section of said hole, respectively, such that said tubes are fixedly positioned in said holes. The working electrode is preferably made of a soft, ductile conductive material such as Cu, Au and Ag. The surface of the perforated plate which constitutes the working electrode is electrically insulated by means of appropriate coating. The working electrode is symmetrically positioned in the space between two counter electrodes that are placed parallel to each other, the distance between said two counter electrodes being preferably in the range of 5 to 8, and more preferably about 6 to 7 cm. Each of the counter electrodes is preferably provided in the form of a plate, or a grid, the area of which being larger than the area of the perforated plate constituting the working electrode. Preferably, the counter electrodes are made of a material selected from the group consisting of Pt Platinized Pt and graphite. The counter electrodes and the working electrode are electrically connected to the positive and negative poles of a power source.

Preferably, in order to assure sufficient concentration of samarium ions in the interior of the tubes placed in the holes of the working electrode, said working electrode is caused to oscillate backwards and forwards to and from each of said counter electrodes in turn, the rate of said oscillatory motion being about 5 to 20 cycles per minute. The oscillatory motion of the working electrode is combined with other modes of mixing of the solution, using, for example, suitable circulation means, which are preferably eductors for pumping and stirring, and filtration means.

A technique known in the art as “Reverse Pulse Plating” (RPP) is advantageously applied, to improve the uniformity of the samarium deposit obtained inside the tubes. The technique is described in CircuiTree, Vol. 14(8), p. 28 (2001) and CircuiTree, Vol. 14(4), p. 52 (2001), which are incorporated herein entirely by reference. Thus, in a preferred embodiment, the method according to the invention described in Israeli patent application no 147199, comprises the steps of:

passing an electrical current of magnitude I_{forward} for a period of time t_{forward}, to electrochemically deposit elemental samarium inside the tubes;

reversing the polarity of the electrodes and passing a reverse current of magnitude Iᵣₑᵥₑᵣₛₑ for a period of time tᵣₑᵥₑᵣₛₑ, wherein I_{forward}<Iᵣₑᵥₑᵣₛₑ and t_{forward}>tᵣₑᵥₑᵣₛₑ,

reversing the polarity of the electrodes,

and repeating said steps to obtain a uniform deposit of samarium inside the tubes.

Preferably, I_{forward} has a current density in the range of 0.5 to 30 mA/cm², and preferably, in the range of 5 to 20 mA/cm². Preferably, the ratio Iᵣₑᵥₑᵣₛₑ:I_{forward} is in the range of 2:1 to 10:1, and preferably about 3:1.

Preferably, t_{forward} is in the range of 10 to 100 msec, and preferably about 40 msec, and tᵣₑᵥₑᵣₛₑ is in the range of 1 to 5 msec, and preferably about 2 to 3 msec.

At the end of the electrodeposition process, the samarium-containing titanium tubes are removed from the working electrode. Following sealing and activation as described above, they are ready for use as brachytherapy seeds.

In a preferred embodiment of the present invention, the implanted radiation sources comprising either ¹⁴⁵Sm or ¹⁷⁰Tm, may, due to the long half-life of said isotopes and also because of their production method, be reactivated following their removal from the body. Optionally, the implanted radiation sources may be marked for the purpose of identification, such that they can be reused in the same patient. Alternatively, the radiation sources may be sterilized such that they can be used for different patients.

The foregoing may be better understood by reference to the following examples, which are provided for illustration purposes.

### EXAMPLES

### Preparation 1

### Preparation of a Radiation Source

Radioactive isotope: ¹⁷⁰Tm,

Casing: titanium tube

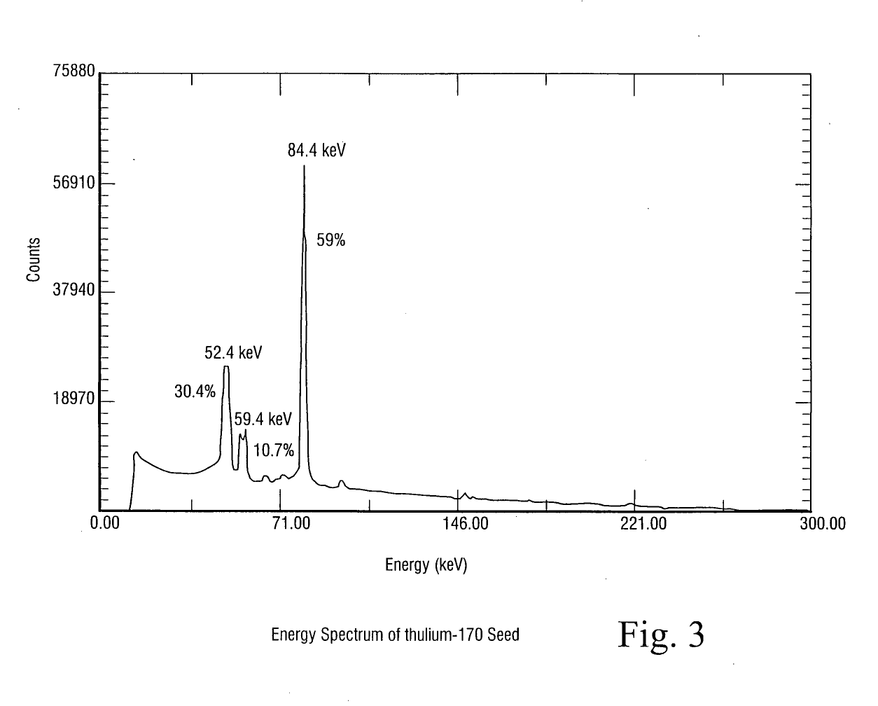

A sheet of ¹⁶⁹Tm having a thickness of 0.2 mm was cut to give tiny, box-like pieces of the following dimensions: 4.5 mm×0.5 mm×0.2 mm. The ¹⁶⁹Tm pieces obtained were inserted into titanium tubes (0.8 mm o.d., 0.7 mm i.d. and 5 mm long, commercially available from Uniform Tubes Inc., 1315 Brunswick Avenue, South Plainfield, N.J. 07080, USA). The total weight of the isotope inserted into the tube was 4.5 mg. Following sealing, the radiation source is activated by means of a neutron flux to convert ¹⁶⁹Tm into ¹⁷⁰Tm. The energy spectrum of ¹⁷⁰Tm is shown in FIG. 3.

### Preparation 2

### Preparation of a Radiation Source

Radioactive isotope: a mixture of ¹²⁵I and ¹²⁷I.

Casing: titanium canister

A mixture containing ¹²⁷I (7 mg, 3.26×10¹⁹ atoms) and ¹²⁵I (60 ng, 2.8×10¹⁴ atoms) was poured into a titanium tube having an inner volume of 1.4×10⁻³ cm³ (0.8 mm o.d., 0.7 mm i.d. and 5 mm long).

### Example I

### Preferential Localization of the In³⁺-tetra(N-methyl-4-pyridyl)-porphyrin) complex within the Nucleus

The following experiment provides a test for selecting particularly useful complexes in accordance with the present invention. The test is based on measuring the number of metal ions that are brought into the malignant cells following the administration of the complexes of the present invention. Particularly useful complexes are defined as those complexes that are capable of bringing more than 10⁵ metal ions into each cell nucleus, and more preferably more than 10⁷ ions into each cell nucleus.

[In³⁺-tetra(N-methyl-4-pyridyl)-porphyrin)] was injected intra-pertioneally into C57 BL mice bearing B16 melanoma on the flank, at a dosage of 40 mg/kg body weight. Tissue samples were taken up to 72 hours after the injection. The samples were treated with trichloroacetic acid (TCA), such that the TCA-insoluble fraction contained the DNA and high molecular weight proteins, and the TCA-soluble fraction contained the cytoplasmic and membrane components of the cells. Inductively-coupled plasma mass spectrometry (ICP-MS) was used to measure the concentration of indium ions in the TCA-insoluble and TCA-soluble fractions.

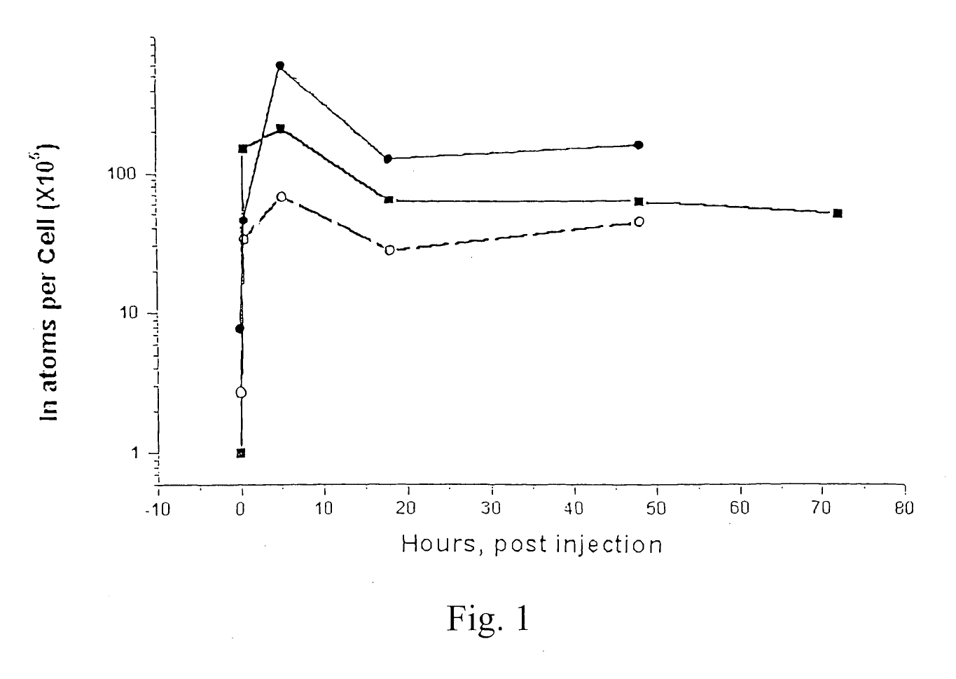

The number of indium ions, per cell, in the TCA-insoluble and TCA-soluble fractions was plotted against time, as shown in FIG. 1. The squares indicate the total number of indium ions (per cell) taken by the tumor, whereas the solid and empty circles indicate the number of indium ions for the TCA-insoluble and TCA-soluble fractions, respectively. It may be seen from the figure that the indium ions carried by the tested complex are preferentially localized in the nucleus, as about 10⁸ to 10⁹ ions of indium (per cell) accumulate in the TCA-insoluble fraction, in comparison to a lesser amount in the cytoplasmic and membrane components. The In atoms localized in tumor cell DNA can be activated by either ¹⁴⁵Sm seeds or by iodine-125 seeds that are available commercially.

### Example II

### Demonstration of the Binding of the In³⁺-tetra(N-methyl-4-pyridyl)-porphyrin) Complex to DNA

The following experiment provides a test for selecting particularly useful complexes in accordance with the present invention on the basis of their capacity to displace ethidium bromide from its binding sites on the DNA molecule.

A stock solution of 1.26 μM ethidium bromide containing 2 mM HEPES, 8 mM sodium chloride and 0.05 mM EDTA (pH=7) was prepared. The solution was used to prepare several samples, and the relative intensity of the fluorescence exhibited by these samples (the wavelengths of the absorption and emission being 546 nm and 598 nm, respectively) was recorded. The compositions of the samples and the relative fluorescent intensity obtained therefor are given in table I.

TABLE I

| | A buffer | | | |
|---|---|---|---|---|
| | solution | | | |
| | containing | | [In³⁺-tetra(N- | Fluorescent |
| Sample | ethidium | DNA | methyl-4-pyridyl)- | Intensity |
| no. | bromide (μl) | (μg) | porphyrin] complex (μg) | (%) |
| 1 | 7.5 | 7.5 | 0.00 | 100 |
| 2 | 7.5 | 7.5 | 0.25 | 85 |
| 3 | 7.5 | 7.5 | 0.50 | 73 |
| 4 | 7.5 | 7.5 | 0.75 | 63 |
| 5 | 7.5 | 7.5 | 1.00 | 54 |
| 6 | 7.5 | 7.5 | 1.25 | 47 |
| 7 | 7.5 | 7.5 | 1.50 | 41 |
| 8 | 7.5 | 7.5 | 1.75 | 35 |
| 9 | 7.5 | 7.5 | 2.00 | 30 |
| 10 | 7.5 | 7.5 | 2.25 | 26 |
| 11 | 7.5 | 7.5 | 2.50 | 22 |
| 12 | 7.5 | 7.5 | 2.75 | 19 |
| 13 | 7.5 | 7.5 | 3.00 | 16 |
| 14 | 7.5 | 7.5 | 3.25 | 13.4 |
| 15 | 7.5 | 7.5 | 3.50 | 12 |
| 16 | 7.5 | 7.5 | 3.75 | 10 |
| 17 | 7.5 | 7.5 | 4.00 | 9 |

* The fluorescent intensity for the stock solution containing no ethidium bromide is 0.

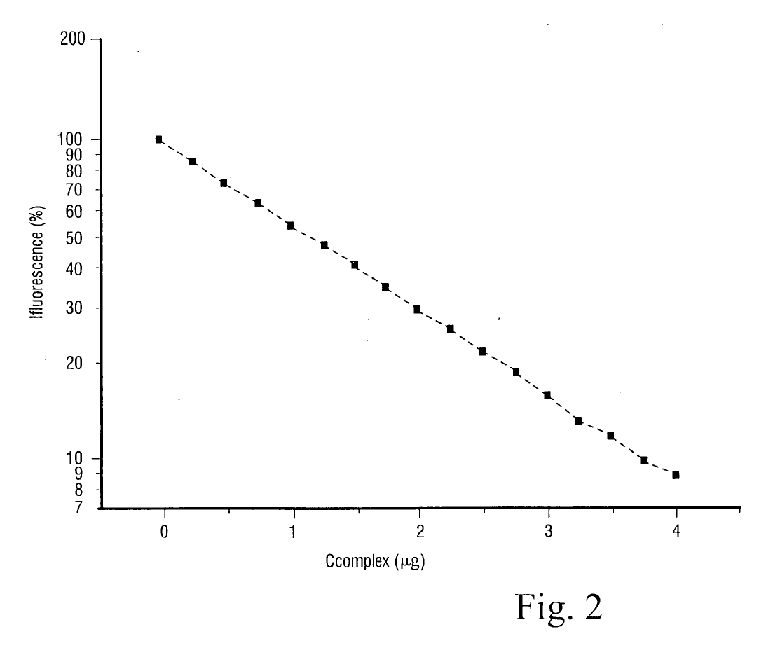

FIG. 2 shows, in a semi-logarithmic scale, a plot of the intensity of the fluorescence exhibited by the samples (designated I_{fluorescence}), against the concentration of the heavy-element containing complex in the samples (designated C_{complex}). It is apparent from the figure that the fluorescence, which is attributed to the bound ethidium bromide, decreases upon increasing the concentration of the complex in the samples (that is, I_{fluorescene} is a decreasing function of C_{complex}). Thus, the In³⁺-tetra(N-methyl-4-pyridyl)-porphyrin displaces the ethidium bromide and becomes bound to the DNA, in what is believed to be an irreversible manner.

### Example III

### Auger Electron Therapy Based on the Use of Pt²⁺-tetra(N-methyl-4-pyridyl)-porphyrin) complex [PtTMPyP(4] in Combination with ¹⁰³Pd Brachytherapy Seed

Materials and Methods:

General description. On day 0, after measurements of the length, width and height of the tumors, palladium-103 brachytherapy seeds were implanted into 12 KHJJ murine mammary carcinoma tumors borne on the thighs of 12 BALB/C mice. Twenty-four hours after seed implantation, 40 mg/kg, platinum (II)-tetrakis(N-methyl-4-pyridyl)porphyrin [PtTMPyP(4)], in the form of its chloride salt, was administered i.p. as a single bolus injection of 200 μl to the six mice undergoing AET. Tumor volume measurements were obtained two to three times weekly. Drug was also administered on days 5 (30 mg/kg), and 19 (20 mg/kg) following seed implantation.

Tumor. Prior to the experiment, tumors were excised from the donor mouse in a sterile environment, and fragmented into 1-2 mm sections in a Petri dish. After anesthetizing each mouse with ketamine/xylazine (10:1), a small slit was prepared through the skin on the thigh, ˜5 mm above and lateral to the distal femur of the right knee. To assure subcutaneous implantation of the tumor fragment, a magnifying glass with a fluorescent light was used to observe the sheen on the fascia surrounding the three sides of the ‘pocket’ produced by the slit. An 18 gauge trochar, into whose bevel a tumor fragment had been inserted, was positioned into the ‘pocket’. A stylette was used to push the fragment into the ‘pocket’. Tumors were implanted either 12 or 15 days prior to the experiment. Upon verification of tumor growth, those tumors of adequate size to accommodate the seed implant, were randomized into treatment groups (AET or radiation only) using a coin flip.

Measurements of tumor volume. Using a digital caliper, the length, width, and height of the tumors were measured. Being ellipsoid in their growth pattern, tumor volume was determined according to the formula, π/6×lwh.

Seed implantation. On day 0, after weighing the mice and measuring the length, width and height of the tumors, palladium-103 brachytherapy seeds (˜2.2 mCi in activity; Theragenics) were implanted into the thigh tumors as follows. The anesthetized mouse was positioned on a board whose angle was ˜45°. The tumor bearing leg was inserted through an elastic band, secured through the board. The foot was taped to the board. An 18 gauge trochar with a funnel head that was supported by a brace on a ring stand, was inserted into the center to the tumor. The seed, previously sterilized by dipping in ETOH, rinsing in a sterile PBS solution, and air-dried, was placed in the funnel of the trochar using long-nosed forceps. A stylette was used to insert the seed into the tumor, after which the trochar was withdrawn from the tumor and Neosporin was applied to the wound.

Drug delivery. The day after seed implantation (day 1), 40 mg/kg of PtTMPyP(4), in the form of its chloride salt, was administered intraperitoneally to the mice in the AET treatment group. On day 5, 30 mg of PtTMPyP(4) was injected i.p., and on day 19, a dosage of 20 mg/kg was injected i.p.

Uptake of Pt atoms in tumor cell DNA. After dissection of the tumor in preparation tumors for inductively-coupled plasma mass spectroscopy (ICP-MS) measurements, the tumor was sectioned into smaller pieces that were added to a pre-weighed tube. A solution of 5% w/v cold TCA was added to the tube, 2.5 ml for each 10 mg of tumor. After vortexing the tube well, it was placed in a centrifuge for 10 min at 3000 rpm, at 4° C. The supernatant was aspirated and placed into another pre-weighed tube for measurement of the TCA soluble fraction containing cellular degradation products. The tube containing the TCA-soluble fraction of the tumor cells was reweighed. Ethanol (100%) was added to the TCA-insoluble fraction (DNA and HMW proteins), 2.5 ml for each 10 mg of weight and the tube was centrifuged again. After aspirating the ETOH, a second similar centrifugation was carried out using 1.2 mg ETOH for very 10 mg of weight, and aspirating the ETOH, a second similar centrifugation was carried out using 1.2 mg ETOH for very 10 mg of weight, and the ETOH aspirated. The TCA-insoluble fraction was dried overnight at 37° C. The tube containing the dried, insoluble fraction was reweighed. One ml of 65% nitric acid (Baker ultra-pure) was added to the TCA soluble fraction, and to the TCA-insoluble fraction, 1 ml of nitric acid was added for every 50 mg of weight. All samples were placed into a hot water bath at 80° C. for 8 hours to digest the biological materials. After digesting the tissues, the samples were centrifuged to assure complete digestion and sent for elemental analysis of platinum. Calculations of the number of platinum atoms per cell in the samples were carried out based upon the weight of each sample, assuming that the weight of the tumor corresponds to 10⁹ cells per mg.

Results

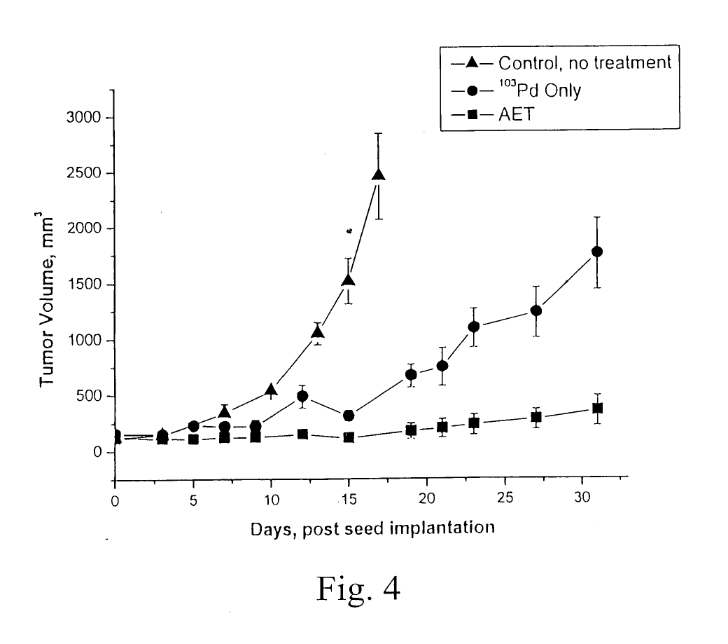

The results are shown in following table and in FIG. 4.

TABLE II

| | | | | Tumor |
|---|---|---|---|---|
| | | | | volume, mm³ ± |
| | | | Tumor | standard error |
| | Tumor | Days, | volume, | Auger Electron |
| | volume, | post | mm³ ± | Therapy: |
| Days, | mm³ ± | seed | standard | 103 Pd seeds in |
| Con- | standard | implan- | error 103 | combination with |
| trol | error | tation | Pd seeds | PtTMPyP(4) |
| 5 | 62.1 ± 54.8 | 0 | 155.9 ± 27.5 | 117.5 ± 18.6 |
| 7 | 162.8 ± 45.4 | 3 | 150.9 ± 2.9 | 112.8 ± 15.3 |
| 11 | 385.5 ± 100.7 | 5 | 228.3 ± 26.0 | 110.6 ± 25.7 |
| 14 | 598.0 ± 159.3 | 7 | 223.0 ± 44.1 | 121.5 ± 29.4 |
| 16 | 782.0 ± 205.5 | 9 | 221.8 ± 58.6 | 123.1 ± 26.9 |
| 18 | 1091.2 ± 210.5 | 12 | 486.2 ± 10.4 | 150.7 ± 31.5 |
| 20 | 1598.1 ± 262.0 | 15 | 312.2 ± 52.6 | 116.5 ± 44.9 |
| 22 | 2202.2 ± 307.3 | 19 | 666.5 ± 103.0 | 175.6 ± 69.2 |
| 24 | 2215.8 ± 273.2 | 21 | 746.1 ± 170.9 | 200.1 ± 81.9 |
| | | 23 | 1093.1 ± 172.1 | 232.6 ± 91.2 |
| | | 27 | 1233.6 ± 225.8 | 280.2 ± 90.6 |
| | | 32 | 1759.2 ± 318.1 | 356.2 ± 133.9 |

The curve shown in appended FIG. 4 represents the mean tumor volume for mice participating in the experimental groups. A significant delay in tumor control can be observed for mice in the AET (Auger electron therapy) treatment group when PtTMPyP(4) was administered compared to those mice that received the radiation alone [¹⁰³Pd] or had no treatment. By day 32, the mean tumor volume of mice with only the brachytherapy seeds reached a volume of ˜1759 mm³, whereas those that had additionally received the drug had a mean growth of ˜356 mm³. This factor of 5 can be attributed to the increase in the radiation dose as a result of the 20 keV photons from ¹⁰³Pd initiating a photoelectric event at the L absorption edge of Pt atom resulting in the dense, highly-local ionizing energy deposition from Auger electrons.

### Example IV

The following section illustrates a preferred embodiment of the therapeutic method according to the present invention for the treatment of prostate cancer. Prior to practicing the method of the present invention, it is preferable to perform three-dimensional imaging of the prostate tumor of the patient to be treated. The morphology of the tumor and its position with regard to surrounding normal tissues may be determined, following which dose calculations may be carried out, to determine the most suitable distribution of energy within the tumor, in order to assure that the radiation dose will be uniformly delivered throughout the tumor. According to the results of said calculations, the desired positioning of the radiation sources (the brachytherapy seeds) in the tumor may be determined. The seeds are then implanted interstitially in the prostate tumor. On the following day, a pre-determined optimal dose of the heavy element-containing complex is administered intravenously to the patient. If required, the drug may be delivered several times during the course of the radiation.

While specific embodiments of the invention have been described for the purpose of illustration, it will be understood that the invention may be carried out in practice by skilled persons with many modifications, variations and adaptations, without departing from its spirit or exceeding the scope of the claims.

1. A method for the treatment of a tumor, comprising administering to a subject a therapeutically effective amount of a complex of a heavy element with a polydentate, pyrrole-containing macrocyclic ligand substituted with charged chemical groups, wherein said complex is capable of bringing said heavy element into close proximity to the nuclear DNA of cells in said tumor, and irradiating said tumor.

2. A method according to claim 1, wherein the tumor is irradiated by means of a radiation source having an energy output capable of activating the heavy element to emit Auger electrons therefrom.

3. A method according to claim 2, wherein the polydentate, pyrrole-containing macrocyclic ligand is a porphyrin substituted with positively charged quaternary ammonium groups or negatively charged carboxylic acid residues.

4. A method according to claim 3, wherein the polydentate, pyrrole-containing macrocyclic ligand is a porphyrin substituted with positively charged quaternary ammonium groups, said quaternary ammonium groups being represented by the following formula: 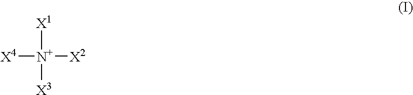
wherein x¹, x², x³ and x⁴ are independently selected from the group consisting of substituted or unsubstituted C₁-C₅ alkyl, C₂-C₅ alkenyl, C₂-C₅ alkynyl, C₃-C₈ carbocyclic radicals, aryl radicals, heterocyclic radicals, heteroaryl radicals, or X and X are taken together with the nitrogen atom to which they are connected to form a heterocyclic radical or heteroaryl radical, wherein, in case of the latter radical, X⁴ is absent.

5. A method according to claim 4, wherein the positively charged quaternary ammonium groups are represented by the following formulas: 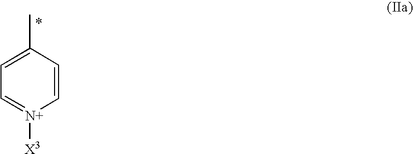
wherein X³ is a straight or branched C₁-C₅ alkyl, and wherein the chemical bond indicated by asterisk signifies the linkage to the porphyrin system; or 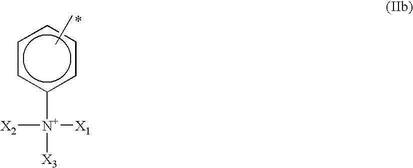
wherein X¹, X² and X³ are straight or branched C₁-C₅ alkyl, and wherein the chemical bond indicated by asterisk signifies the linkage to the porphyrin system.

6. A method according to claim 3, wherein the complex of the heavy element with the polydentate, pyrrole-containing macrocyclic ligand is metalloporphyrin represented by the structure of formula III: 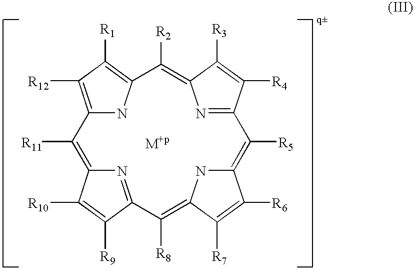
wherein M^{p+} designates a cation of the heavy element capable of exhibiting the Auger effect, q± represents the total charge of the complex, which may be either positive or negative, and wherein:
(i) R₂, R₅, R₈ and R₁₁ are positively charged N-alkyl pyridyls of the formula 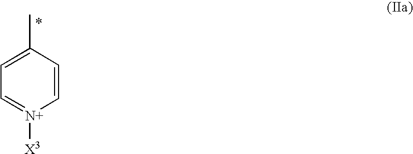
wherein X³ is a straight or branched C₁-C₅ alkyl and the chemical bond indicated by asterisk signifies the linkage to the porphyrin system of formula III, and R₁, R₃, R₄, R₆, R₇, R₉, R₁₀ and R₁₂ are hydrogen; or
(ii) R₂, R₅, R₈ and R₁₁ are positively charged N,N, N-trialkyl anillinium of the formula 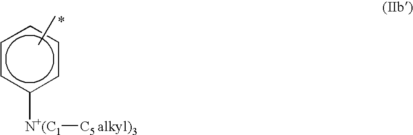
wherein the bond indicated by asterisk signifies the linkage to the porphyrin system, and R₁, R₃, R₄, R₆, R₇, R₉, R₁₀ and R₁₂ are hydrogen; or
(iii) R₃, R₆, R₁₀ and R₁₂ are methyl groups, R₇ and R₉ are negatively charged carboxylic acid residues —(CH₂)n-C(0)0⁻, wherein n is an integer between 1-5, R₁ and R⁴ are represented by the formula: 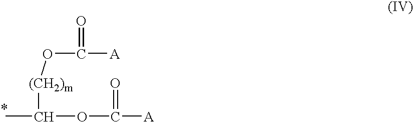
wherein m is an integer between 1-5, A is fullerene (C₆₀), and the chemical bond indicated by asterisk signifies the linkage to the porphyrin system of formula III, and wherein R₂, R₅, R₈ and R₁₁ are hydrogen.

7. A method according to claim 6, wherein the heavy element containing complex is selected from the group of M^{p+}-tetra (N-alkyl-4-pyridyl)porphyrins.

8. A method according to claim 1, wherein the heavy element is selected from the group consisting of In³⁺, Gd³⁺, Pt²⁺, Pd²⁺ and Au³⁺.

9. A method according to claim 8, wherein the complex is selected from the group consisting of:
In³⁺-tetrakis(N-methyl-4-pyridyl)porphyrin
In³⁺-tetrakis(4-N,N,N-trimethylanilinium)porphyrin
In³⁺-tetrakis-fullerene-carboxylate ester of 2,4 bis
(α,β-dihydroxyethyl)-deutroporphyrin IX.
Pt²⁺-tetrakis(N-methyl-4-pyridyl)-porphyrin).

10. A method according to claim 1, wherein the radiation source is implanted near or at the body region to be treated, said radiation source comprising one or more radioactive isotopes that are packed within a casing provided in the form of a closed, cylindrically shaped, canister.

11. A method according to claim 10, wherein the implanted radiation source comprises ¹²⁵I, or a mixture of ¹²⁵I and ¹²⁷I.

12. A method according to claim 10, wherein the implanted radiation source comprises ¹⁷⁰Tm.

13. A method according to claim 10, wherein the heavy element containing complex is Pt²⁺-tetra(N-methyl-4-pyridyl)-porphyrin) and the implanted radiation source comprises ¹⁰³Pd.

14. A method according to claim 10, wherein the radiation source is in the form of a canister containing ¹⁴⁵Sm, wherein said canister is produced by the following steps:
providing a solution containing samarium (¹⁴⁴Sm) ions;
positioning a working electrode and at least one counter electrode in contact with said solution;
connecting said working electrode and said at least one counter electrode to the negative and positive poles of a power source, respectively;
passing an electrical current between said electrodes to electrochemically deposit elemental samarium on said working electrode in a geometrical form corresponding to the form of the interior of the canister; concurrently or sequentially loading said canister with said elemental samarium; and
neutron-irradiating said elemental ¹⁴⁴Sm, to produce the radioactive ¹⁴⁵Sm.

15. A therapeutic composition for use in Auger radiation therapy of tumors, comprising a complex of a heavy element with a polydentate, pyrrole-containing macrocyclic ligand substituted with charged chemical groups, wherein said complex is capable of bringing said heavy element into close proximity to the nuclear DNA of cells in said tumors, and wherein said heavy element is capable of emitting Auger electrons, together with a pharmaceutically acceptable carrier.

16. A therapeutic composition according to claim 15, comprising a complex of a heavy element with porphyrin substituted with positively charged chemical groups, for use in radiation therapy of tumors.

17. A therapeutic composition according to claim 16, comprising a complex of a heavy element with porphyrin substituted with positively charged quaternary ammonium groups, for use in radiation therapy of tumors.

18. A therapeutic composition according to claim 17, comprising a complex of a heavy element with porphyrin substituted with positively charged quaternary ammonium groups, wherein said quaternary ammonium groups are represented by the following formula: 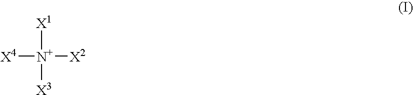
wherein X¹, X², X³ and X⁴ are independently selected from the group consisting of substituted or unsubstituted C₁-C₅ alkyl, C₂-C₅ alkenyl, C₂-C₅ alkynyl, C₃-C₈ carbocyclic radicals, aryl radicals, heterocyclic radicals, heteroaryl radicals, or X¹ and X² are taken together with the nitrogen atom to which they are connected to form a heterocyclic radical or heteroaryl radical, wherein, in case of the latter radical, X⁴ is absent.

19. A therapeutic composition according to claim 18, wherein the positively charged quaternary ammonium groups are represented by the following formulas: 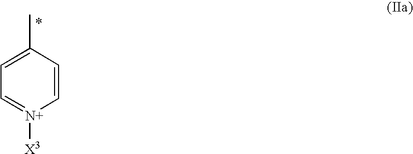
wherein X³ is a straight or branched C₁-C₅ alkyl, and wherein the chemical bond indicated by asterisk signifies the linkage to the porphyrin system; or 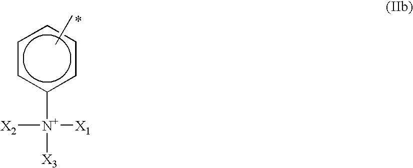
wherein X¹, X² and X³ are straight or branched C₁-C₅ alkyl, and wherein the chemical bond indicated by asterisk signifies the linkage to the porphyrin system.

20. A therapeutic composition according to claim 17, wherein the complex of the heavy element with the polydentate, pyrrole-containing macrocyclic ligand is metalloporphyrin represented by the structure of formula III: 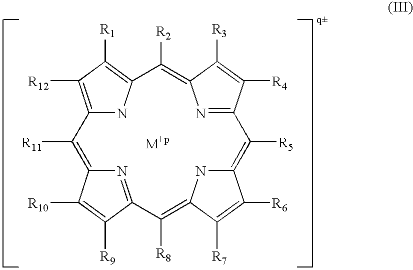
wherein M^{p+} designates a cation of the heavy element capable of exhibiting the Auger effect, q± represents the total charge of the complex, which may be either positive or negative, and wherein:
(i) R₂, R₅, R₈ and R₁₁ are positively charged N-alkyl pyridyls of the formula 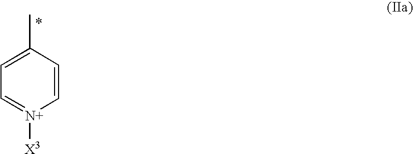
wherein X³ is a straight or branched C₁-C₅ alkyl and the chemical bond indicated by asterisk signifies the linkage to the porphyrin system of formula III, and wherein R₁, R₃, R₄, R₆, R₇, R₉, R₁₀ and R₁₂ are hydrogen; or
(ii) R₂, R₅, R₈ and R₁₁ are positively charged N,N, N-trialkyl anillinium of the formula 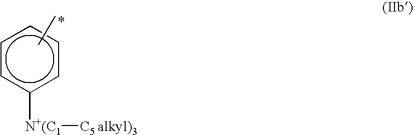
and the bond indicated by asterisk signifies the linkage to the porphyrin system of formula III and wherein R₁, R₃, R₄, R₆, R₇, R₉, R₁₀ and R₁₂ are hydrogen; or
(iii) R₃, R₆, R₁₀ and R₁₂ are methyl groups, R₇ and R₉ are negatively charged carboxylic acid residues —(CH₂)n-C(0)0⁻, wherein n is an integer between 1-5, R₁ and R₄ are represented by the formula: 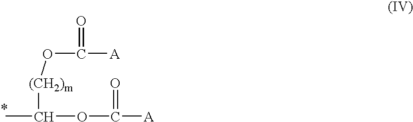
wherein m is an integer between 1-5, A is fullerene (C₆₀), and the chemical bond indicated by asterisk signifies the linkage to the porphyrin system of formula III, and wherein R₂, R₅, R₈ and R₁₁ are hydrogen.

21. A therapeutic composition according to claim 20, wherein the heavy element containing complex is selected from the group of M^{p+}-tetra(N-alkyl-4-pyridyl)porphyrins.

22. A therapeutic composition according to claim 20, wherein the cation of the heavy element, M^{p+}, is selected from the group consisting of In³⁺, Gd³⁺, Pt²⁺ and Au³⁺.

23. A therapeutic composition according to claim 22, wherein the complex is selected from the group consisting of:
In³⁺-tetrakis(N-methyl-4-pyridyl)porphyrin
In³⁺-tetrakis(4-N,N,N-trimethylanilinium)porphyrin
In³⁺-tetrakis-fullerene-carboxylate ester of 2,4 bis
(α,β-dihydroxyethyl)-deutroporphyrin IX.
Pt²⁺-tetrakis(N-methyl-4-pyridyl)-porphyrin).

24. Use of a complex of a heavy element with a polydentate, pyrrole-containing macrocyclic ligand substituted with charged chemical groups in the preparation of a medicament useful in radiation therapy of tumors, wherein said complex is capable of bringing said heavy element into close proximity to the nuclear DNA of cells in said tumor, and wherein said heavy element is capable of emitting Auger electrons.

25. Use of a complex according to claim 24, wherein the polydentate, pyrrole-containing macrocyclic ligand is substituted with positively charged chemical groups.

26. Use of a complex according to claim 25, wherein the positively charged substituents are quaternary ammonium groups.

27. Use according to claim 26, wherein the complex is a complex of a heavy element with porphyrin substituted with positively charged quaternary ammonium groups, wherein said quaternary ammonium groups are represented by the following formula: 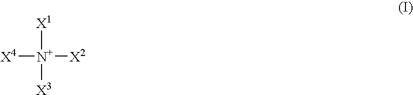
wherein X¹, X², X³ and X⁴ are independently selected from the group consisting of substituted or unsubstituted C₁-C₅ alkyl, C₂-C₅ alkenyl, C₂-C₅ alkynyl, C₃-C₈ carbocyclic radicals, aryl radicals, heterocyclic radicals, heteroaryl radicals, or X¹ and X² are taken together with the nitrogen atom to which they are connected to form a heterocyclic radical or heteroaryl radical, wherein, in case of the latter radical, X⁴ is absent.

28. Use according to claim 27, wherein the positively charged quaternary ammonium groups are represented by the following formulas: 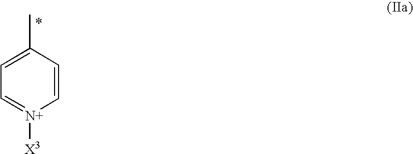
wherein X³ is a straight or branched C₁-C₅ alkyl, and wherein the chemical bond indicated by asterisk signifies the linkage to the porphyrin system; or 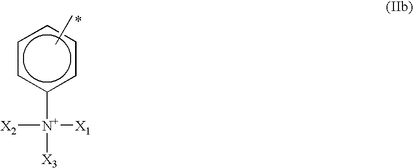
wherein X¹, X² and X³ are straight or branched C₁-C₅ alkyl, and wherein the chemical bond indicated by asterisk signifies the linkage to the porphyrin system.

29. Use according to claim 26, wherein the complex of the heavy element with the polydentate, pyrrole-containing macrocyclic ligand is metalloporphyrin represented by the structure of formula III: 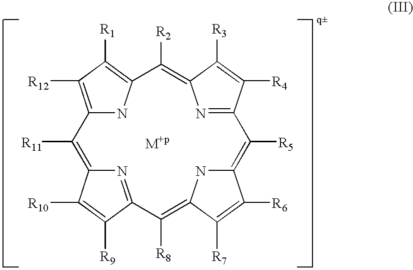
wherein M^{p+} designates a cation of the heavy element capable of exhibiting the Auger effect, q± represents the total charge of the complex, which may be either positive or negative, and wherein:
(i) R₂, R₅, R₈ and R₁₁ are positively charged N-alkyl pyridyls of the formula 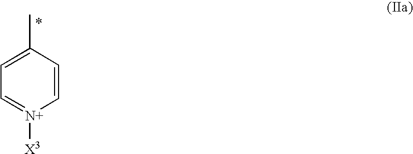
wherein X³ is a straight or branched C₁-C₅ alkyl and the chemical bond indicated by asterisk signifies the linkage to the porphyrin system of formula III, and wherein R₁, R₃, R₄, R₆, R₇, R₉, R₁₀ and R₁₂ are hydrogen; or
(ii) R₂, R₅, R₈ and R₁₁ are positively charged N,N, N-trialkyl anillinium of the formula 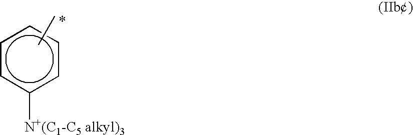
and the bond indicated by asterisk signifies the linkage to the porphyrin system of formula III and wherein R₁, R₃, R₄, R₆, R₇, R₉, R₁₀ and R₁₂ are hydrogen; or
(iii) R₃, R₆, R₁₀ and R₁₂ are methyl groups, R₇ and R₉ are negatively charged carboxylic acid residues —(CH₂)n-C(0)0⁻, wherein n is an integer between 1-5, R₁ and R₄ are represented by the formula 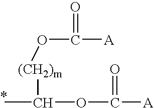
wherein m is an integer between 1-5, A is fullerene (C₆₀), and the chemical bond indicated by asterisk signifies the linkage to the porphyrin system of formula III, and wherein R₂, R₅, R₈ and R₁₁ are hydrogen.

30. Use according to claim 29, wherein the heavy element containing complex is selected from the group of M^{p+}-tetra(N-alkyl-4-pyridyl)porphyrins.

31. Use according to claim 29, wherein the cation of the heavy element, M^{p+}, is selected from the group consisting of In³⁺, Gd³⁺, Pt²⁺ and Au³⁺.

32. A therapeutic system suitable for the radiation therapy of tumors, comprising:
a therapeutic composition according to claim 15; and
a radiation source to irradiate said tumor having an energy output capable of activating the heavy element to emit Auger electrons therefrom.

33. A therapeutic system according to claim 32, wherein the radiation source is provided in the form of a radioactive isotope packed in an implantable, cylindrically shaped, canister.

34. A therapeutic system according to claim 33, wherein the heavy element is selected from the group consisting of indium, platinum, gold and gadolinium, and the polydentate, pyrrole-containing macrocyclic ligand substituted with charged chemical groups is porphyrin substituted with positively charged quaternary ammonium groups.

35. An implantable radiation source comprising a titanium canister containing ¹⁷⁰Tm.

36. A process for preparing ¹⁷⁰Tm-containing implantable radiation source, comprising loading titanium tube with ¹⁶⁹Tm and converting said ¹⁶⁹Tm into ¹⁷⁰Tm.

37. A radiation source comprising a mixture of ¹²¹I and ¹²⁷I.
